(19) United States
(12) Patent Application Publication
(10) Pub. No.: US 2012/0172617 A1
(43) Pub. Date: Jul. 5, 2012

(54) RESVERATROL INTERMEDIATES DAL

(76) Inventors: Werner Bonrath, Freiburg (DE); Ulla Letinois, Saint-Louis (FR); Reinhard Karge, Staufen (DE); Max Hugentobler, Arlesheim (CH)

(21) Appl. No.: 13/143,413

(22) PCT Filed: Jan. 6, 2010
(86) PCT No.: PCT/EP2010/000021

§ 371 Date: Feb. 29, 2012

(30) Foreign Application Priority Data
Jan. 6, 2009 (EP) 09000065.4

(51) Int. Cl. C07C 67/28; C07C 69/18

(52) U.S. Cl. 560/144

(57) ABSTRACT

A process for the preparation of 1-(3,5-diacetoxyphenyl)-ethanol by catalytic hydrogenation of 3,5-diacetoxy-acetophenone in the presence of a Ni-alloy as catalyst in a C₁₋₃-carboxylic acid ester as solvent.

Resveratrol, 3,4′,5-trihydroxy-stilbene, is a phytoalexin naturally produced by several plants when under attack by pathogens such as bacteria or fungi. Resveratrol has attracted increasing interest in view of the health benefits which have been reported such as cardiovascular-protective, anti-cancer, antiviral, anti-aging, and anti-inflammatory activity. Resveratrol is available in form of extracts from natural sources, e.g. giant knotweed or red grapes, or in high purity as a synthetically prepared chemical.

Resveratrol is obtained, e.g., by a multi-step chemical synthesis from commercially available 3,5-diacetoxy-acetophenone (DAK) as described in WO 2005/023740. In the first step of this synthesis DAK is hydrogenated catalytically over platinum (5%, w/w) on charcoal in tetrahydrofuran (THF) or methanol to the corresponding alcohol, 1-(3,5-diacetoxy)-ethanol (DAL). After evaporation of the solvent the brown oil obtained was purified by flash-chromatography on silica gel using n-hexane/ethyl acetate which yielded DAL as colorless oil in a purity of 98% (GC). It has been stated that compounds like DAK can be reduced to form a compound like DAL by catalytic hydrogenation, e.g., using a noble metal catalyst, such as Pd or Pt on charcoal, or an activated Ni catalyst such as Raney Ni, in alcoholic, e.g., methanolic solution.

The application of noble metal catalysts at long reaction times results in an increased formation of by-products.

In the reduction of DAK to DAL by batch-wise hydrogenation of a 10 wt % solution of DAK in THF at 70° C. under 10 bar in the presence of 5 wt % Pt/C₁-(3-acetoxyphenyl)-ethanol (APE), 1-(3-acetoxy-5-hydroxy)-phenyl-ethanol (AHPE) and (3,5-diacetoxy)-1-phenyl-1-acetoxyethane (DPA) were identified as by-products. The aim of the present work was to by-pass the disadvantages of state-of-the-art procedures. It has now surprisingly been found that when using a nickel-alloyl catalyst in an acetic acid ester the hydrogenation proceeds with good reproducibility in a shorter period of time with less by-products, e.g. less than 1% of the critical by-product APE. The results are surprisingly better than in alcoholic solution.

Therefore, the present invention relates to a new process for the preparation of 1-(3,5-diacetoxyphenyl)-ethanol by catalytically hydrogenating 3,5-diacetoxy-acetophenone with a Ni-alloy in an acetic acid ester, preferably methyl or ethyl ester and to the (3,5-diacetoxyphenyl)-ethanol thus obtained or obtainable.

The advantages of the new process are higher selectivity, lower waste and much easier separation of the catalyst from the reaction medium.

Any Ni-alloy known to be useful as catalytic hydrogenation catalyst which is commercially available and offered by several companies, e.g., Evonik, can be used in the reaction of the present invention. Apart from Ni in an amount of at least 90 weight-%, preferably at least 93 weight-% or at least 95 weight-%, the alloy may contain other metals, e.g., Al, Fe, Cr, Mo and Co. The preferred Ni-alloy is Raney Ni. Examples of suitable Ni-alloy catalysts have the following composition: Ni 90-95%, Al 5.5-8%, Fe<0.4%, Mo<0.01%, Cr<0.03%

The reaction is carried out conveniently under conditions well-known to the person skilled in the art, i.e., under a hydrogen pressure of 0.1-50 bar, preferably 0.3-20 bar and more preferably 0.5-5 bar, at a temperature in the range of 60-100° C., until 100% conversion has been achieved. The reaction mixture is worked-up and the DAL is isolated in pure form in accordance with methods known in the art.

The invention is illustrated in more detail by the following examples.

### EXAMPLE 1

1.8 g of Ni-alloy Degussa B 113 Z (humid) were weighed into a 500 ml stainless steel autoclave fitted with a gassing stirrer. 235.0 g of ethyl acetate and 100.0 g of 3′,5′-diacetoxy-acetophenone were added and the autoclave was closed. The mixture was stirred at 500 rpm and the autoclave was flushed three times with 5 bar nitrogen. The stirrer was then stopped and the autoclave was flushed twice with 3 bar hydrogen for the elimination if nitrogen.

During ten minutes a pressure test with 5 bar hydrogen was carried out. The stirrer was started again to 500 rpm and the autoclave was heated to 70° C.

When the internal temperature had reached 70° C., the stirrer was stopped and the autoclave was pressurized to 2 bar hydrogen. Then the stirrer was set to 1000 rpm and the course of the reaction was monitored by in-process-control.

After 20 hours the autoclave was cooled to 20° C. The reaction mixture was filtrated over a membrane filter (0.45 mm) and the autoclave was washed with ethyl acetate. After evaporation of the solvent from the crude reaction mixture the yield of DAL was 97%, the amount of the critical by-product ACE was 0.1%.

### EXAMPLE 2

25 mg of nickel-alloy Degussa B 113 Z (humid) were weighed in an 8 ml Carius tube, fitted with a magnetic stirring bar. 1 ml of isopropanol and 250 mg of 3′,5′-diacetoxy-acetophenone were added and the Carius tube was closed. The mixture was stirred at 500 rpm and the Carius tube was flushed three times with 5 bar nitrogen. Stirring was then stopped. The Carius tube was flushed twice with 3 bar hydrogen for the elimination of nitrogen.

During ten minutes a pressure test with 5 bar hydrogen was carried out.

Stirring was started again to 500 rpm and the Carius tube was heated to 70° C. When the internal temperature had reached 70° C., stirring was stopped and the Carius tube was pressurized to 2 bar hydrogen.

Then the stirrer was set to 1000 rpm and the course of the reaction was monitored by in-process-control. After 20 hours the Carius tube was cooled to 20° C. The reaction mixture was filtrated over a membrane filter (0.45 mm) and the Carius tube was then washed with ethyl acetate. After evaporation of the solvent from the crude reaction mixture the yield of DAL was 92%, the critical by-product ACE was not detected.

Results of experiments carried out under variation of pressure, catalyst, solvent and recycling following the above procedure can be found in the Tables 1, 2 and 4, respectively, below.

### EXAMPLE 3

A 30 wt % solution of 15 g of DAK was hydrogenated in ethyl acetate in the presence of Ni-alloy or Pt/C at 70° C. The results obtained after 24 hours are summarized in Table 1.

| | Amount | Pressure | | Yield | By products | |
|---|---|---|---|---|---|---|
| | DAK | H₂ | Catalyst | DAL | ACE | all others |
| No. | [g] | [bar] | [g] | [%] | [%] | [%] |
| 1 | 15.0 | 10 | Ni-alloy | 98.05 | 1.83 | n.d. |
| | | | 1.77 | | | |
| 2 | 15.0 | 10 | Ni-alloy | 98.39 | n.d. | 1.6 |
| | | | 1.75 | | | |
| 3 | 15.0 | 2 | Ni-alloy | 97.85 | 0.13 | 2.01 |
| | | | 1.89 | | | |
| 4 | 15.0 | 2 | Ni-alloy | 97.86 | 0.14 | 2.00 |
| | | | 1.80 | | | |
| 5 | 15.0 | 2 | Ni-alloy | 96.97 | 0.13 | 2.90 |
| | | | 1.80 | | | |
| 6 | 15.0 | 2 | Pt/C | 91.04 | 0.39 | 2.6 |
| | | | 1.50 | | | |

### EXAMPLE 4

500 mg of DAK in 1.2 g of different solvents were hydrogenated under 10 bar hydrogen pressure at 70° C. in the presence of 150 mg of Ni-alloy or 500 mg of 5% Pt/C or Pd/C during 24 hours. The results are summarized in Table 2.

| No. | Solvent | Catalyst | Conversion [%] | Yield product [%] |
|---|---|---|---|---|
| 1 | EtOAc | Ni-alloy | 100 | 95.0 |
| 2 | | Pt/C 5% | 100 | 91.7 |
| 3 | | Pd/C 5% | 99 | 34.3 |
| 4 | THF | Ni-alloy | 100 | 94.4 |
| 5 | | Pt/C 5% | 99 | 96.8 |
| 6 | | Pd/C 5% | 100 | 73.6 |
| 7 | 2-Propanol | Ni-alloy | 100 | 92.1 |
| 8 | | Pt/C 5% | 100 | 12.8 |
| 9 | | Pd/C 5% | 99 | 22.7 |
| 10 | water | Ni-alloy | 100 | 76.7 |
| 11 | | Pt/C 5% | 99 | 44.3 |
| 12 | | Pd/C 5% | 100 | 36.1 |

By-products were determined in experiments on larger scale (≧100 g starting material) and compared to a batch from the pilot plant. In Table 3 the formation of by-products APE, AHPE and DPA depending on the conversion of DAK is summarized:

| Entry | Time | Catalyst/Conditions | DAK | APE | AHPE | DPA |
|---|---|---|---|---|---|---|
| Pilot | n.d. | Pt/C, THF, 10 bar | 0.2 | 0.41 | 2.0 | 0.63 |
| plant | | | | | | |
| 1 | 20 h | Ni-alloy, EtOAc, 2 bar | 0.8 | 0.12 | 2.4 | 0.8 |
| 2 | 8 h | Ni-alloy, EtOAc, 2 bar | 0.8 | 0.05 | 2.7 | 0.9 |
| 3 | 4 h | Ni-alloy, EtOAc, 2 bar; | 2.8 | 0.14 | 1.6 | 0.17 |
| | | 3-fold amount | | | | |
| 4 | 21 h | Pt/C, EtOAc, 2 bar | 4.9 | 0.51 | 0.4 | 0.19 |
| 5 | 6 h | Pt/C, THF, 2 bar | 19 | 0.42 | 0.5 | n.d. |

Using Ni-alloy the amount of APE is always lower than in the hydrogenation over Pt/C, even at higher conversion. The amount of AHPE is higher than over Pt/C, but this is not critical, as AHPE reacts later to the desired product.

### EXAMPLE 5

For the recycling test, 15 g of DAK were hydrogenated in 35 g of ethyl acetate over 5.8 g of Nickel-alloy under 2 bar hydrogen pressure at 70° C. The reactions were stopped after 120% of the theoretical hydrogen uptake. After each run the reaction mixture was sucked out of the reaction vessel, the catalyst remaining in the autoclave. The catalyst was not washed; it was reused as it was for the next run. The results are shown in Table 4:

| No. | Catalyst | Time [h] | Yield |
|---|---|---|---|
| Cycle 1 | Ni-alloy | 19.5 | 92.6 |
| Cycle 2 | Ni-alloy (Cycle 1) | 15.5 | 95.3 |
| Cycle 3 | Ni-alloy (Cycle 2) | 19 | 95.2 |
| Cycle 4 | Ni-alloy (Cycle 3) | 19 | 96.2 |

The results show that Ni-alloy can be re-used from the first run for at least four following runs without affecting the yield. The occurring by-products were identical to those usually encountered in this reaction (see Table 3, entries 1, 2 and 3).

1. A process for the preparation of 1-(3,5-diacetoxyphenyl)-ethanol by catalytically hydrogenating 3,5-diacetoxy-acetophenone with a Ni-alloy, characterized in that the reaction is carried out in a C₁₋₃-carboxylic acid ester.

2. The process of claim 1, wherein the C₁₋₃-carboxylic acid ester is a C₁₋₄-alkyl ester.

3. The process of claim 1, wherein the C₁₋₃-carboxylic acid ester is acetic acid methyl ester or acetic acid ethyl ester.

4. The process according to claim 1, wherein the transformation of 3,5-diacetoxy-acetophenone is at least 99% (w/w).

5. A process according to claim 1, wherein the yield of 1-(3,5-diacetoxyphenyl)-ethanol is at least 96% (w/w).

6. A process according to claim 1, wherein the sum of by-products is below 2.0% (w/w).

7. A process according to claim 1, wherein the critical by-product 1-(3-acetoxyphenyl)-ethanol is generated in less than 1% (w/w).

8. A process according to claim 1, wherein the reaction is carried out in ethyl acetate as solvent.

9. 1-(3,5-diacetoxyphenyl)-ethanol obtained or obtainable according to a process according to claim 1.
